(19) United States
(12) Patent Application Publication
(10) Pub. No.: US 2025/0135216 A1
(43) Pub. Date: May 1, 2025

(54) Electronic Controls for Generating Shaped RF Magnetic Fields for In Vivo Disinfection of Surfaces of Electrically Conductive Medical Devices, Parts and Components

(71) Applicants: John C. Tepper, Carollton, TX (US); Varun Sadaphal, Dallas, TX (US); James Kevin McCoy, Garland, TX (US); Jared A. Smotherman, Addison, TX (US)

(72) Inventors: John C. Tepper, Carollton, TX (US); Varun Sadaphal, Dallas, TX (US); James Kevin McCoy, Garland, TX (US); Jared A. Smotherman, Addison, TX (US)

(21) Appl. No.: 18/934,322

(22) Filed: Nov. 1, 2024

Related U.S. Application Data
(60) Provisional application No. 63/595,256, filed on Nov. 1, 2023.

(51) Int. Cl. A61N 1/40; A61L 2/00; A61L 2/24; A61N 1/08

(52) U.S. Cl. CPC A61N 1/406; A61L 2/0023; A61L 2/24; A61N 1/08; A61L 2202/14; A61L 2202/16; A61L 2202/21

(57) ABSTRACT

An electronic control system for a generator of alternating magnetic fields for in vivo disinfection of electrically conductive surfaces of medical implants, which may be used with a wide variety of transducer heads and appliances. The electronic control system includes an impedance matching function which maximizes power transfer from a signal source to the transducer heads dynamically according to the load placed within the field produced by the transducer coils.

This application claims benefit of the filing date of U.S. Provisional Patent Application 63/595,256, filed on Nov. 11, 2023, by John C. Tepper. This invention is related to systems and methods for disinfecting surfaces of electrically conductive medical devices, parts and components which have been implanted internally to patients, including but not limited to metallic implants and carbon fiber implants.

### FIELD OF THE INVENTION

### Background of the Invention

There are a wide variety of metallic, metal-based and carbon fiber medical implants used in medicine today. Prosthetic joints, artery and vein stents, fracture plates, bone anchors, dental implants, etc., all contain sofme metallic parts that contact living tissue. There is a susceptibility of device surfaces to infection by a bacterial biofilm on the metal surfaces. Surgery, oral antibiotics and intravenous antibiotics have had limited success combating these bacterial biofilm infections.

### SUMMARY OF THE INVENTION

An electronic control system is disclosed for a generator of alternating magnetic fields for in vivo disinfection of electrically conductive surfaces of medical implants, which may be used with a wide variety of transducer heads and appliances. The electronic control system includes an impedance matching function which maximizes power transfer from a signal source to the transducer heads dynamically according to the load placed within the field produced by the transducer coils.

### BRIEF DESCRIPTION OF DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIG. 1 shows a functional schematic diagram for electronic controls of a system according to at least one embodiment of the present invention.

FIG. 2 depicts a perspective view of example embodiment of a treatment system according to the present invention.

### DETAILED DESCRIPTION OF THE DISCLOSED EMBODIMENT(S) OF THE INVENTION

An alternating magnetic field (AMF) is a non-invasive approach to treat infections of implanted medical devices, such as knee or hip implants. Previous patents have disclosed early attempt to provide an external transducer coil which generates time-varying AMF in the vicinity of a metal implant. The AMF induces surface electrical currents on the implant's electrically conductive portions, which can heat the surface of the implant. In the case of an infected implant, bacteria (which may be in the form of a biofilm) adheres to the surface within this heated area on the surface, which can eradicate pathogens or sensitize them to additional antimicrobial treatments such as antibiotics. One such patent is WO2022/140226 A1, published on 30 Jun. 2022, by the World Intellectual Property Organization (WIPO) by inventors David Greenberg, et al., for applicant Solenic Medical Inc. of Texas, United States.

Reference will now be made to the drawings wherein like structures may be provided with like suffix reference designations. In order to show the structures of various embodiments more clearly, the drawings included herein are diagrammatic representations of structures. Thus, the actual appearance of the fabricated structures, for example in a photo, may appear different while still incorporating the claimed structures of the illustrated embodiments (e.g., walls may not be exactly orthogonal to one another in actual fabricated devices). Moreover, the drawings may only show the structures useful to understand the illustrated embodiments. Additional structures known in the art may not have been included to maintain the clarity of the drawings. For example, not every layer of a device is necessarily shown. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. “Connected” may indicate elements are in direct physical or electrical contact with each other and “coupled” may indicate elements cooperate or interact with each other, but they may or may not be in direct physical or electrical contact. Phrases such as “comprising at least one of A or B” include situations with A, B, or A and B. Further, throughout this disclosure, “metallic surface” is used to refer collectively to all types of electrically conductive surfaces, including but not limited to carbon fiber surfaces.

Applicant determined precise positioning of the AMF transducer coil(s) outside of the patient and relative to the target metal implant within the patient is important for accurate surface interaction (e.g., heating) of the implant. Applicant determined misalignment of the implant (i.e., incorrect positioning of the implant relative to the transducer coil(s)) can result in under or over exposure of the implant, which could impact the safety and efficacy of the treatment. User error and patient-to-patient variability need to be mitigated to achieve treatment within a safe envelope of operation. Because the implant is not visible in the non-invasive AMF treatment, Applicant determined a method and system is needed to ensure repeatable and precise positioning of the coil or coils with respect to the implant.

Still further, Applicant determined that the performance of a simple RF magnetic field produced in a center or core of a cylindrical-shaped transducer could be significantly improved in efficiency of treating in vivo infections on surfaces of implants and medical devices by shaping the field to conform it closely to the contour of the infected metallic surface which would also trduce unwanted heating of nearby tissue.

Even further, Applicant determined that, upon shaping the field to correspond to the metallic surface to be treated, the energy level of the field could be increased significantly, thereby further increasing the efficacy of the non-intrusive AMF treatment machine and process.

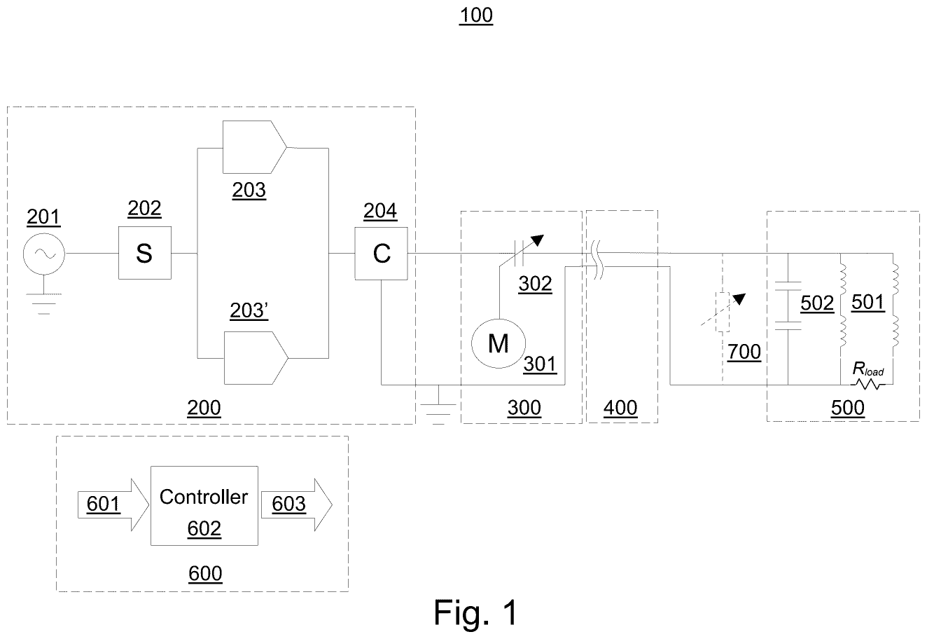

Turning now to FIG. 1, a functional schematic diagram 100 for electronic controls of a system according to at least one embodiment of the present invention is shown. The system can be divided for reference purposes only into several sections 200, 300, 400, 500 and 600. It will be readily understood by those ordinarily skilled in the relevant arts that this grouping of sections does not limit the integration of some electronic components with others, nor does it limit the separation of some electronic components into multiple components, in other embodiments according to the present invention.

A signal generating section 200 provides an alternating electronic signal to an impedance matching section 300, which then transmits significant power of a variable frequency, voltage and current through an interconnect section 400 to a shaped-field transducer 500 which is, during treatments, aligned with and positioned around an implant to be treated. A controller section 600 receives inputs from monitoring sensors, measuring sensors, and user interfaces, and generates control signals to various components and subsystems to accomplish the advanced functionality as described herein.

Signal Generation Section. The signal generating section 200 includes a signal source 201 which creates a low-power alternating signal Varive of a small voltage amplitude and limited current output to a splitter 202. The power of the signal source's output can be determined using a conventional computation such as a root-mean-squared formula for alternating sinusoidal-shaped signals. For the particular example embodiment according to the present invention, a variable frequency of about 216 kHz is produced.

The splitter 202 sends signals having half of the power of the received Varive signal to each of two amplifiers 203, 203′. The splitter 202 preferably maintains phase alignment of all of its outputs to the amplifiers. Some other embodiments may utilize just one amplifier, in which case a splitter would not be necessary. In other embodiments, multiple N amplifiers may be utilized, and in such embodiments, N-way splitters may be utilized. In other embodiments, an active splitter may be utilized which outputs multiple signals of the same power as it receives from the signal source.

The amplifiers 203, 203′, are preferably of a type which have a predictable phase delay from their input to their output so that all of the amplifier output signals are phase aligned, and can be combined by a signal combiner 204, thereby combining all of the power of all of the amplified signals into a single, high-power alternating signal to be output to the impedance matching section 300.

In at least one prototype, the following components were used:
a. a custom low-voltage sinusoidal signal generator circuit for a signal source 201, outputting V_{drive} signal according to frequency, amplitude and an on/off control signals from the control section 600;
b. a Model SLF-2 available from Electronics and Innovation, LTD, (E&I) of Rochester, New York, for a splitter 202, receiving V_{drive} from the signal source 201, and having two phase-aligned outputs to the two amplifiers 203 and 203′;
c. a Model 100S04 available from Electronics and Innovation, LTD, (E&I) of Rochester, New York, for each 1 KW linear amplifier 203, 203′, receiving the split signal from the splitter 202, each amplifier providing +60 dB gain, into an output impedance of 50Ω, and providing monitoring signals Vₘₒₙ and Iₘₒₙ to the controller section 600, wherein the Vₘₒₙ signal presents 1 V for every 50 V output and the Iₘₒₙ signal presents 1 V for every 50 A output; and
d. a Model CLF-2 available from Electronics and Innovation, LTD, (E&I) of Rochester, New York, for the combiner 204, which as a 50Ω input and which combines the phase-aligned amplified signals into an excitation signal of up to 9 A into the impedance matching section 300.

Impedance Matching Section. The output of the combiner 204 can be said to be “looking into” the combined impedances of the impedance matching section 300, interconnect section 400 connected to a shaped-field transducer 500, ultimately driving power to an “RF load”, the implant 700, which is physically within the alternating magnetic field of the transducer 500. It should be noted that Applicant has determined that the apparent RF impedance of the transducer 500 with its field-coupled implant 700 changes impedance based on the presence (or not) of a portion of a patient with a metallic implant. The impedance presented to the output of the combiner 204 may be changed by changing the frequency of the RF signal source 201 and the capacitance of the variable matching capacitor 302, in the following way: a control process operating in the Controller 602 continuously varies the frequency of the signal source 201 in order to cause a condition of “zero reactive power”, wherein the RF voltages and currents from the two RF amplifiers 203 and 203′ are in-phase, and thus only real power is being delivered to the matching capacitor, etc. A Controller process continuously monitors the Vₘₒₙ and Iₘₒₙ signals provided by the power amplifiers in order to determine the relative phase of these RF voltages and currents.

Furthermore, during a pre-treatment, low-power “check phase”, another controller process will adjust the capacitance of the variable matching capacitor (by sending control commands to the motor/driver 301 in order to drive the impedance looking into the matching capacitor to be close to the preferred operating point of the power amplifiers and combiner (normally around 500 real). Furthermore, another control process continuously adjusts the signal level of the signal source 201 so that the delivered real power (Vₘₒₙ×Iₘₒₙ for both amplifiers) stays close to the specified pre-treatment or treatment power level.

Whereas the Applicant determined that it is possible to deliver much greater AMF power to the surface of the implant when the field is shaped to correspond to the surface to be treated, it is also an object of the present invention to deliver as much of the power of the excitation signal to the transducer as possible. However, Applicant determined that because the impedance looking into the combined interconnect section 400 and shaped-field transducer 500 changes according to the presence or absence of a metallic implant, Applicant devised a novel approach to matching the impedance of the source of the excitation signal dynamically to match that of the interconnect section 400 combined with the shaped-field transducer 500.

In at least one embodiment, this is accomplished with a large variable capacitor 302 which is operated mechanically via rotation of a shaft via a motor 301 which is controlled by the controller section 600. The controller section 600, or a suitable alternative control circuit, can compare the amount of power being generated via (e.g., Vₘₒₙ×Iₘₒₙ at the amplifiers) to the power being received by the transducers to determine if the real-time impedance of the impedance of the interconnect section 400 combined with the shaped-field transducer 500 is currently matched. If not, the motor can be activated to rotate one direction or the other direction to increase or decrease capacitance, thereby increasing or decreasing the internal impedance of the source of the signal (e.g., section 100 combined with section 200) to match the real-time impedance of the combination of the interconnect and transducers.

In at least one prototype, the following components were used:
a. a Nema 23 stepper motor (Bipolar L=56 mm with gear ration 4:1 planetary gearbox) available from StepperOnline of Nanjing City, Jiangsu Province, China, and a model TMCM-1260 stepper motor controller/driver module available from Analog Devices of Wilmington, Massachusetts, to rotate the shaft of the variable capacitor, having forward, reverse and stop control signal inputs received from the controller section 600; and
b. a variable vacuum capacitor model CVP-1500-40S (100-1500 pF) available from Greenstone USA Inc. of Fremont, California, for the variable capacitor.

In some embodiments, the motor may have a shaft position encoder to allow sensing by the controller section of the rotation and extremes of the shaft to enable more advanced control processes to be implemented by the firmware or software of the controller section, controller circuit, or equivalent controller section.

Interconnect Section. Because the transducers, referred to as the Treatment Transducer Coil Assembly (TTCA), must be co-located with the patient but the signal generation and tuning sections may be located several feet from the TTCA, the interconnect section 400 performs an important function of conveying the high-power, high-voltage signal from the signal generation and tuning sections to the TTCA with the transducers 500. It is currently envisioned that the TTCA will be mounted on a positioning arm which will assist with the alignment and holding of the transducer section in place during treatments, and will hold the TTCA conveniently away from the treatment area between treatments to allow the patient and technician freedom of movement in the area.

As such, the interconnect section can, in some embodiments, include the wires and conductors that run between the output of the tuning section to the beginning of any positioning arm, if present, and may also include the wires and conductors that pass alongside or within the positioning arm. In other embodiments, the wires and cables passing alongside or within the positioning arm may be considered part of the TTCA or even part of the arm itself (if the arm is categorized as a separate section of its own). Those skilled in the relevant arts will readily recognize that inclusion of such functions in one section or another is within ordinary design options, and all such embodiment variations are within the scope and spirit of the present invention.

Cables and wires should be designed in conventional manners for safety, EMI/EMC requirements, trip hazards, overhead plenum air return requirements, and pinch and wear protection. In this particular embodiment, multiple sensor measurements are made by the hardware and firmware in the TTCA and communicated to the control section 600 over a serial data protocol and an electrical (physical) interface specifically compatible with RS-422 twisted pair standards. In this particular embodiment the following measurements are collected and communicated to the control section 600 about five (5) times per second:
a. Left-side and right-side coil temperatures;
b. High-side and low-side tank capacitor temperatures;
c. “Coupon” temperature;
d. Left-side and right-side coil RMS currents; and
e. Left-side and right-side TTCA contact switch open/closed status.

Transducer Section. The electronics of the transducers are included in the transducer section 500, and in some embodiments, the mechanical positioning arm (“gantry”) may be considered to be part of this section as might be the mechanical components and elements for routing the transducer windings to produce the desired shaped field. For the purposes of this disclosure and the example embodiment of the present invention, the mechanical details of the positioning arm and of the windings mechanical housing will be provided in separate sections.

The transducer section 500 nominally includes the high-voltage, high-current windings for creating and emitting an RF magnetic field at the center of which an in vivo medical implant may be treated. The large inductive windings 501 and one or more impedance and tank capacitors 502 of the field generating and emitting portion of the TTCA, as shown in FIG. 1, wherein the implant to be treated 700 appears as a series resistance R_{load} when the implant is present within the volume formed by the windings 501.

For various intended treatments, different embodiments may include different windings, with different induction values, and with different three-dimensional arrangements of the windings to produce different field shapes.

In at least one embodiment, fiber optic temperature sensors are placed in strategic locations within the transducer section in order to allow the controller section to monitor heating of the elements of the transducer section

In the particular example embodiment which is intended for treating a knee replacement, twelve (12) series-connected polyester film power capacitors model CSM 150/300 (0.17 μF each) available from Celem Passive Components Ltd. of Jerusalem, Israel, for the tank capacitors 502.

In this particular example embodiment of the present invention, the circulating current during excitation and generation of the RF magnetic field is about 140 Arms (for a treatment power of 1200 W), with 14 nF of tank capacitance and 37 μH of tank inductance. This corresponds to a tank voltage of about 7 kVrms Other values are possible for other TTCAs to treat other in vivo medical appliances such as hip replacements, elbow replacements, shoulder replacements, heart stents, peripheral artery stents, bone pins and fracture plates.

Coupon Sensor. In at least one embodiment according to the present invention, a “coupon” is a small piece of stainless steel, measuring about 18×18×3 mm which is placed in a convenient, fixed location within the TTCA, and to which one of the fiber-optic temperature sensors is affixed. The idea is that the coupon will get enough of the RF magnetic field to heat up enough during a treatment (AT about 15° C. after 90 seconds of an ˜1200 W maximum power treatment) to provide the controller section with another indirect measurement of RF magnetic field strength within the transducer coil assembly.

Controller Section. The controller section 600 includes, in this example embodiment, a model STM32H723 microcontroller available from ST Microelectronics of Plan-les-Ouates of Switzerland. In the TTCA, a remote sensor board used a model MSP430FR2476 available from Texas Instruments of Dallas, Texas. The controller section receives the aforementioned sensor and monitor signals, such as Vₘₒₙ and Iₘₒₙ, and produces the aforementioned control signals, such as V_{drive} in at least one embodiment, using microcontroller firmware to implement the logic and control equations of the system.

TTCA Windings and Housing. A variety of treatment heads, a.k.a. TTCA, can be driven by the control system disclosed herein. It is expected that several different TTCA designs may be useful in order to optimize the treatment of various types of implants due to the different sizes and shapes of implants (e.g., knees, hips, elbows, shoulders, pins, plates) and the different sizes and shapes of human body portions in which those implants may exist.

Positioning (Gantry) Arm. It is expected that some treatment heads may be heavy and/or clumsy to move manually, and alignment of the treatment head with the in vivo implant to be treated may be greatly assisted by a gantry or positioning and placement arm. Such an arm could use a variety of weight offsetting mechanisms, such as springs, counterweights, cables, and even motors, to reduce the strength needed of a technician to move the treatment head into place for treatment and to move it into a stowing position between treatments. Such a gantry can also be used to route and hold the wires and fiber optic conductors of the interconnect section of the system, thereby producing a medical appliance user-experience similar to that of an x-ray machine or an overhead light.

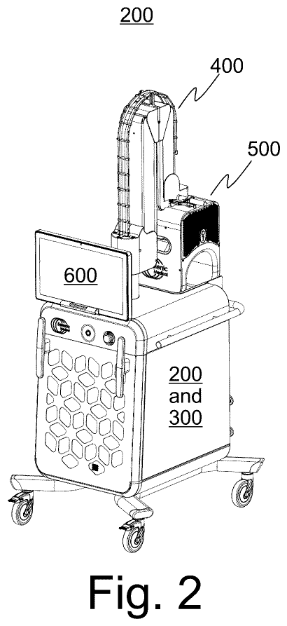

Turning now to FIG. 2, an example embodiment of a system 200 according to the present invention is shown in perspective view. The signal generation section 200 and the impedance matching section 300 are contained in a main housing, which also has a user interface with the controller section 600. The interconnect section 400 and a gantry arm connect the main housing to the TTCA 500, which is shown in this view stowed on the back of the main housing. However, during treatment, the TTCA will be removed from its stowed position, and moved to a position around or near the knee implant to be treated, causing the gantry 400 to extend from it's folded position. The narrow side-to-side design of the main housing is useful, whereas it allows the unit to be positioned alongside a table or bed where the patient is located, while also allowing enough space between the main housing and the table or bed for a technician, nurse, or other operator to move between the unit and the bed or table, depending on the length of the gantry.

Conclusion. The foregoing description of the embodiments of the invention has been presented for the purposes of illustration and description. It is not intended to be exhaustive or to limit the invention to the precise forms disclosed. This description includes terms, such as left, right, top, bottom, over, under, upper, lower, first, second, etc. that are used for descriptive purposes only and are not to be construed as limiting. For example, terms designating relative vertical position refer to a situation where a side of a substrate is the “top” surface of that substrate; the substrate may actually be in any orientation so that a “top” side of a substrate may be lower than the “bottom” side in a standard terrestrial frame of reference and still fall within the meaning of the term “top.” The term “on” as used herein does not indicate that a first layer “on” a second layer is directly on and in immediate contact with the second layer unless such is specifically stated; there may be a third layer or other structure between the first layer and the second layer on the first layer.

The terminology used herein is for the purpose of describing particular exemplary embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof, unless specifically stated otherwise.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Certain embodiments utilizing a microprocessor executing a logical process may also be realized through customized electronic circuitry performing the same logical process(es). The foregoing example embodiments do not define the extent or scope of the present invention, but instead are provided as illustrations of how to make and use at least one embodiment of the invention.

The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

I/We claim:

1. A control process for a system for disinfecting electrically conductive surfaces of medical devices, parts and components which have been implanted in a patient body, wherein the system has one or more electromagnetic-field shaping transducers for generating induction heating, the control process comprising:
receiving two or more inputs representing a complex power transfer from outputs of one or more signal amplifiers into a combination of an impedance matching section, an interconnect section and a transducer section of the system; and
reducing a reactive power component of the complex power transfer by dynamically varying one or more outputs selected from the group consisting of a frequency selection control output of a signal source feeding the one or more signal amplifiers, an amplitude control output of a signal source feeding the one or more signal amplifiers, a variable capacitance control output of the impedance matching section, and a variable inductance control output of the impedance matching section.

2. The control process as set forth in claim 1 wherein the electrically conductive surface comprises a metallic surface.

3. The control process as set forth in claim 1 wherein the receiving two or more inputs representing a complex power transfer comprises receiving a voltage-monitor input electrically connected to a voltage-monitor output of the one or more power amplifiers, wherein the power amplifier is amplifying an excitation signal from a signal source.

4. The control process as set forth in claim 1 wherein the receiving two or more inputs representing a complex power transfer comprises receiving a current-monitor input electrically connected to a current-monitor output of the one or more power amplifiers, wherein the power amplifier is amplifying an excitation signal from a signal source.

5. The control process as set forth in claim 4 wherein the excitation signal comprises a sinusoidal signal at a specific frequency and at a specific amplitude.

6. The control process as set forth in claim 1 wherein the dynamic varying of the one or more outputs is performed, at least in part, according to an electrically conductive load received within the electromagnetic field generated by the transducers.

7. The control process as set forth in claim 1 wherein the reducing a reactive power component of the complex power transfer by dynamically varying one or more outputs further comprises:
determining a phase difference between the received receiving two or more inputs; and
generating the frequency control output to vary a frequency of the signal source until the phase difference is minimized below a pre-determined threshold.

8. The control process as set forth in claim 1 wherein the reducing a reactive power component of the complex power transfer by dynamically varying one or more outputs further comprises selecting an optimal operating point of the one or more power amplifiers.

9. The control process as set forth in claim 1 wherein the reducing the a reactive power component of the complex power transfer comprises varying the one or more outputs to achieve an impedance of approximately 50 ohms looking into the combination of the impedance matching section, the interconnect section and the transducer section of the system.

10. The control process as set forth in claim 1 further comprising controlling a real power component of the complex power transfer to the one or more electromagnetic-field shaping transducers to achieve a specific treatment level by dynamically varying one or more outputs selected from the group consisting of a frequency selection control output of a signal source feeding the one or more signal amplifiers, an amplitude control output of a signal source feeding the one or more signal amplifiers, a variable capacitance control output of the impedance matching section, and a variable inductance control output of the impedance matching section.

11. A controller for a system for disinfecting electrically conductive surfaces of medical devices, parts and components which have been implanted in a patient body, wherein the system has one or more electromagnetic-field shaping transducers for generating induction heating, the controller comprising:
a processor for executing program instructions;
a computer-readable memory device which is not a propagating signal per se; and
one or more program instructions encoded by the computer-readable memory device which, when executed by the processor, perform the steps comprising:
receiving two or more inputs representing a complex power transfer from outputs of one or more signal amplifiers into a combination of an impedance matching section, an interconnect section and a transducer section of the system; and
reducing a reactive power component of the complex power transfer by dynamically varying one or more outputs selected from the group consisting of a frequency selection control output of a signal source feeding the one or more signal amplifiers, an amplitude control output of a signal source feeding the one or more signal amplifiers, a variable capacitance control output of the impedance matching section, and a variable inductance control output of the impedance matching section.

12. The controller as set forth in claim 11 wherein the receiving two or more inputs representing a complex power transfer comprises receiving a voltage-monitor input electrically connected to a voltage-monitor output of the one or more power amplifiers, wherein the power amplifier is amplifying an excitation signal from a signal source.

13. The controller as set forth in claim 11 wherein the receiving two or more inputs representing a complex power transfer comprises receiving a current-monitor input electrically connected to a current-monitor output of the one or more power amplifiers, wherein the power amplifier is amplifying an excitation signal from a signal source.

14. The controller as set forth in claim 11 wherein the dynamic varying of the one or more outputs is performed, at least in part, according to an electrically conductive load received within the electromagnetic field generated by the transducers.

15. The controller as set forth in claim 11 wherein the reducing a reactive power component of the complex power transfer by dynamically varying one or more outputs further comprises:
determining a phase difference between the received receiving two or more inputs; and
generating the frequency control output to vary a frequency of the signal source until the phase difference is minimized below a pre-determined threshold.

16. The controller as set forth in claim 11 wherein the reducing a reactive power component of the complex power transfer by dynamically varying one or more outputs further comprises selecting an optimal operating point of the one or more power amplifiers.

17. The controller as set forth in claim 11 wherein the instructions for reducing a reactive power component of the complex power transfer comprises varying the one or more outputs to achieve an impedance of approximately 50 ohms looking into the combination of the impedance matching section, the interconnect section and the transducer section of the system.

18. The controller as set forth in claim 11 wherein the instructions further comprise instructions for controlling a real power component of the complex power transfer to the one or more electromagnetic-field shaping transducers to achieve a specific treatment level by dynamically varying one or more outputs selected from the group consisting of a frequency selection control output of a signal source feeding the one or more signal amplifiers, an amplitude control output of a signal source feeding the one or more signal amplifiers, a variable capacitance control output of the impedance matching section, and a variable inductance control output of the impedance matching section.
